(19) United States
(12) Patent Application Publication
(10) Pub. No.: US 2006/0134598 A1
(43) Pub. Date: Jun. 22, 2006

(54) Cell culture media dispenser

(75) Inventor: James W. Kenney, Broomall, PA (US)

Correspondence Address: JOSEPH M. KONIECZNY, P.C., PO Box 387, CONSHOHOCKEN, PA, 19428-0387, US

(73) Assignee: Drummond Scientific Company, Broomall, PA (US)

(21) Appl. No.: 11/017,144

(22) Filed: Dec. 20, 2004

(51) Int. Cl. C12Q 3/00; C12M 1/36

(52) U.S. Cl. 435/3; 435/286.4; 435/286.5; 435/287.3

(57) ABSTRACT

An apparatus and method of dispensing precise aliquots of cell culture media from a bulk, sterile, flexible container. The apparatus has load cell, a control valve, and a programmable controller. The container suspends from the load cell within the housing. The load cell continuously transmits to the controller an output signal, which represents the force, i.e., weight, of the bag on the load cell. The controller regulates the flow of media from the container by selectively activating the control valve. The controller precisely measures the volume of fluid media dispensed from the container by continuously calculating the change in weight of the container using the load cell output signal.

### FIELD OF THE INVENTION

The present invention relates to an apparatus and method for dispensing precise amounts of fluid from a container. More particularly, the invention relates to an apparatus and method for dispensing precise aliquots of cell culture media from a bulk, sterile, flexible container.

### BACKGROUND OF THE INVENTION

A cell repository, or cell bank, is a collection of living cells maintained indefinitely at extremely low temperatures in a state of suspended animation. Cell repositories, such as the Coriell Institute, establish, maintain, and distribute thousands of different cell lines/cultures, which are used by scientists worldwide for scientific and clinical research. Typically, each cell line is indexed in a searchable database. Upon request, a vial of the specified cell line is recovered from cold storage, thawed, and shipped to the requesting scientist.

To grow a cell line in the repository or laboratory, the cells must be “fed” with cell culture media. The composition of cell culture media varies depending on the cell line with which the media is to be used. At cell repositories, each particular batch of cell culture media is prepared from bulk quantities, typically a 20-liter flexible bag, of base media, to which particular constituents are added to satisfy a particular cell line. The media is then apportioned into much smaller glass or plastic bottles prior to use at the repository or shipment to customers.

During cell line feeding, the cell culture media is typically dispensed from the bottles using a pipetter equipped with a disposable pipet. Each time the pipet is immersed in the media, a risk of contamination occurs if the pipet is not sterile or if contaminants enter through the open top of the bottle. Further, a risk of cross contamination between cell lines occurs if the same media bottle is used to feed several different cell lines. If contamination occurs, both the cell culture media and the cell line must be discarded.

To reduce the risk of cross contamination, laboratory protocol typically limits use of each media bottle to 3 or less distinct cell lines. Because of the risk of contamination, the volume of cell culture media containers is typically limited to low volumes, such as ¼-liter bottles, so that potentially large batches of media are not wasted (discarded) if contamination occurs.

Packaging cell culture media in low-volume units significantly increases the overall cost per unit of media. For example, the material cost of multiple low-volume bottles is much higher than the material cost of a single large bag. Likewise, the shipping and handling cost of multiple low-volume bottles is much higher than the shipping cost of a single, large bag.

The disposal cost of multiple small-volume bottles is also much higher than a large bag because multiple, low-volume bottles weigh more and are bulkier. The disposal cost of cell culture media containers is amplified because such containers are classified as bio-hazardous waste, which is significantly more expensive to discard than non-hazardous waste.

### SUMMARY OF THE INVENTION

The invention provides an apparatus and method for dispensing precise amounts of fluid from a container. The apparatus and method have particular use for dispensing precise aliquots of cell culture media from a flexible container having a flexible dispense tube.

The apparatus generally includes a housing, load cell, control valve, and programmable controller. The container suspends from the load cell within the housing. The load cell continuously transmits to the controller an output signal, which represents the force, i.e., weight, of the bag on the load cell. The controller regulates the flow of media from the container by selectively activating the control valve. The controller precisely measures the volume of fluid media dispensed from the container by continuously calculating the change in weight of the container using the load cell output signal.

The control valve regulates fluid flow through the dispense tube. Preferably, the control valve comprises a solenoid-actuated pinch valve, which engages only the outer surface of the flexible dispense tube and does not contact the cell culture media.

In one embodiment, fluid is dispensed from the container simply by opening the control valve and allowing gravity to force fluid from the container. In another embodiment, the dispenser includes a container pressurizer for increasing fluid flow from the container. The container pressurizer may comprise a pneumatic compression sleeve, which surrounds and compresses the container. A pneumatic pump, which is functionally connected to the controller, pressurizes the sleeve. The compression sleeve preferably suspends from the load cell and supports the container therein.

In another embodiment, the housing is fully-enclosed and can be either positively or negatively pressurized by a pump in fluid communication therewith. Positively pressurizing the housing increases fluid flow from the container. Negatively pressurizing the housing allows fluid to be infused into the bag through the dispense tube.

The controller can be programmed to dispense aliquots of any volume less than or equal to the volume (V) of the container. The controller can also be calibrated with the specific density of the media contained within the container.

The controller preferably includes a digital microprocessor, an amplifier, and an analog-to-digital converter (ADC). The ADC preferably comprises either a successive-approximation (SAR) ADC amplifier integrated circuit or a sigma-delta ADC amplifier integrated circuit. The controller may also include one or more trigger switches, a keypad and an LCD display.

To prevent contamination of the media in the container during dispensing, the apparatus includes a flexible extension tube releasably fixed to the free end of the dispense tube of the container. A disposable pipet is releasably fixed to the end of the extension tube. If either the pipet or extension tube is contaminated, it can be replaced without contaminating the fluid contained in the container.

In a preferred embodiment of the method of the invention, the container is suspended from a load cell. The initial weight of the container is calculated based on the load cell measurement. The target weight of the container, i.e., the weight after the aliquot is dispensed, is also calculated. Fluid is dispensed from the container while continuously monitoring the actual weight of the container. Once the actual container weight equals the target weight, fluid dispense is terminated. In the preferred embodiment, fluid dispense is controlled by intermittently restricting flow through the flexible dispense tube. The container may be externally pressurized to increase fluid flow from or into the container.

### BRIEF DESCRIPTION OF THE DRAWINGS

FIG. 1 is a front elevational view of a dispenser apparatus in accordance with an embodiment of the invention shown attached to the side panel of a laminar flow hood;

FIG. 1A is an enlarged, front elevational view of the control valve of the apparatus of FIG. 1;

FIG. 2 is an enlarged front elevational view of the dispenser housing and internal components of the apparatus of FIG. 1;

FIG. 3 is a schematic diagram of the dispenser apparatus of FIG. 1;

FIG. 4 is a logic diagram of the controller of the apparatus of FIG. 1;

FIG. 5 is a front elevational view of a dispenser apparatus in accordance with another embodiment of the invention;

FIG. 6 is an enlarged front elevational of the dispenser housing and internal components of the apparatus of FIG. 5;

FIG. 7 is a front plan view of a dispenser housing in accordance with a further embodiment of the invention; and,

FIG. 8 is a schematic diagram of the controller of the apparatus of FIG. 7.

### DETAILED DESCRIPTION OF PREFERRED EMBODIMENTS

For the purpose of illustration, there is shown in the accompanying drawings several embodiments of the invention. The apparatus and method are described below with particular application to dispensing precise, controlled amounts of cell culture media for the purpose of feeding cell lines in a sterile environment. However, it should be appreciated that the apparatus and method are useful for dispensing precise, controlled amounts of a variety of fluids in various environments. Further, it should be understood by those of ordinary skill in the art that the apparatus and method are not limited to the precise arrangements and instrumentalities shown herein and described below.

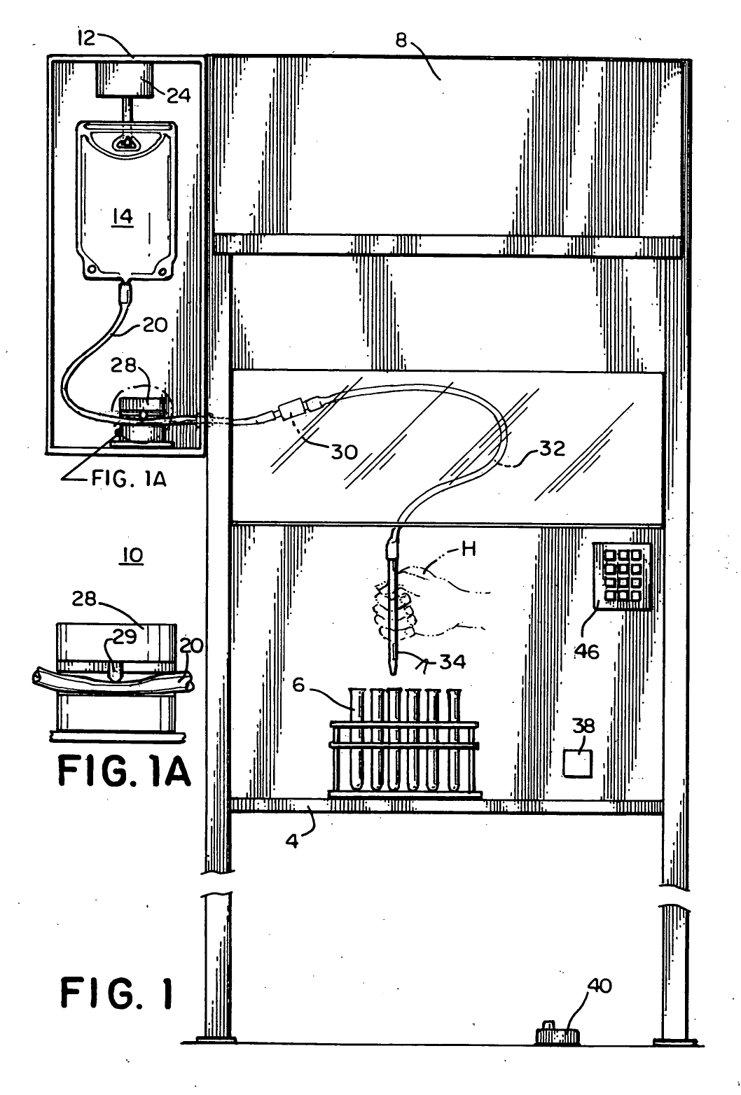

A cell culture media dispenser in accordance with an embodiment of the invention is shown in FIGS. 1-4, and is designated generally by reference numeral 10. The apparatus 10 dispenses precise aliquots of cell culture media from a disposable, bulk container. As used herein, the term “aliquot” shall mean a defined volume of fluid that is repeatedly dispensed until such volume is redefined. In FIG. 1, the dispenser 10 is shown mounted to a laminar flow hood 8 having a bench top 4, which supports a plurality of test tubes 6, or other containers, housing the cell lines that require feeding. While the feeding is preferably conducted inside the hood 8, the dispenser 10 and container need not be located therein.

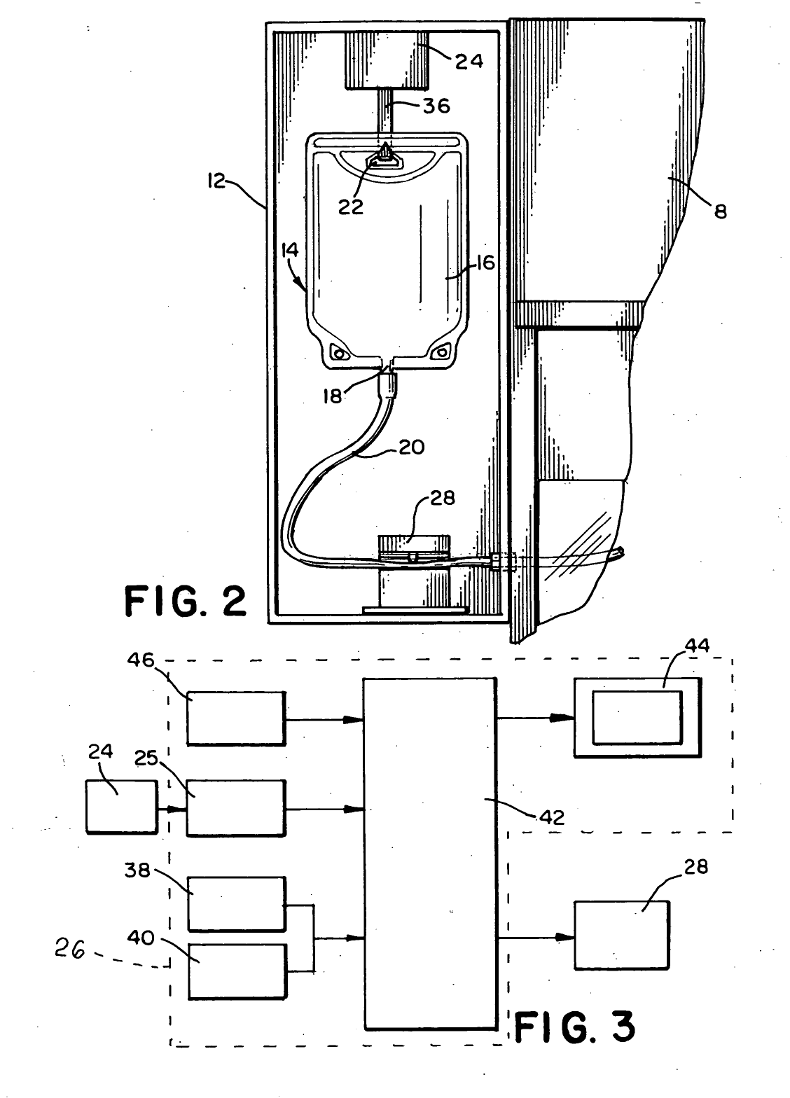

In the embodiment shown in FIGS. 1-4, the container comprises a polymer bag 14 having a storage reservoir 16, an outlet 18 at the bottom end, and a flexible dispense tube 20 connected to the outlet 18. Referring to FIG. 2, the bag 14 preferably has a molded opening or eyelet 22 in the upper periphery. The bag 14 may comprise, for example, a standard, 2-liter, flexible ethyl vinyl acetate (EVA) container such as sold under the trademark Secure™ by The Metrix Company, Dubuque, Iowa. However, it should by appreciated to those of ordinary skill in that art that other forms and sizes of bulk container could be used without departing from the invention.

The dispenser 10 may include a housing 12, which may be mounted to the laminar flow hood 8 or another support surface proximate the flow hood 8. In the embodiment illustrated in FIGS. 1-4, fluid is expelled from the bag 14 by the force of gravity; therefore, the bag 14 and housing 12 should be mounted at an elevated position relative to the bench top 4 and the cell cultures resting thereon. To protect the bag 14 and other components, the housing 12 may be enclosed with a door or cover, not shown.

A device for continuously weighing the bag 14 is fixed in the housing 12. In a preferred embodiment, the weighing device comprises a load cell 24 fixed to the upper portion of the housing 12. The load cell 24 includes a support hook 36, which engages the eyelet 22 of the bag 14 and freely suspends the flexible bag 14 within the housing 12.

In the embodiment shown in FIGS. 1-4, the load cell 24 comprises a model S300 overload-proof, single-point load cell manufactured by Strain Management Devices, Meridian, Conn. The load cell includes four strain gauge sensor elements, which are attached to a mechanical suspension beam and connected electrically in a bridge arrangement. With suitable electrical excitation, the load cell 24 develops an electrical output signal, which corresponds to the force acting on the suspension beam, i.e., the weight of the flexible bag 14.

A control valve is also preferably located within the housing 12. The control valve regulates media flow from the bag 14. In a preferred embodiment, the control valve comprises a pinch valve 28, which engages only the outer surface of the dispense tube 20 and does not contact the media flowing through the tube 20. For example, the pinch valve 28 may comprise model number 360P011-42 NC manufactured by NResearch, Maplewood N.J. As best seen in FIG. 1A, the pinch valve 28 includes a plunger 29, which releasably compresses the dispense tube 20 without piercing or severing the dispense tube wall, thereby eliminating the pinch valve 28 as a contamination source. The pinch valve 28 is preferably located proximate the load cell 24 and acts as an anchor to prevent the dispense tube 20 from “tugging” on the load cell 24.

If a pinch valve 28 is selected as the control valve, the dispense tube 20 should comprise a durable, elastomeric material, which will withstand repetitive compression and release of the dispense tube 20 until the bag 14 is empty. For this reason, the dispense tube 20 is preferably made from silicone or C-flex tubing.

The load cell 24 and pinch valve 28 are functionally connected to a programmable controller 26. Within the tolerances of the dispenser 10, the controller 26 can be programmed and calibrated to dispense a precise aliquot of any volume less than or equal to the volume (V) of the bag 14.

The controller 26 preferably includes a readily-available, programmable microprocessor 42. For example, the microprocessor 42 may comprise a programmable microprocessor such as model AT89S8252 manufactured by Amtel, Inc., Agoura Hills, Calif.

Referring to FIG. 3, the controller 26 also preferably includes a keypad 46 and an LCD display 44 connected to the microprocessor 42. The keypad 46 enables the user to activate, calibrate, and program the dispenser 10. The LCD 44 displays a variety of modes and information such as aliquot volume, current bag weight, bag volume lower limit, calibration status, etc. The controller 26 may also contain one or more activation switches. For example, the controller 26 may include a finger trigger switch 38 or foot switch 40, whose output signal (“trigger signal”) is taken by the controller 26 as an instruction to initiate dispense of an aliquot. A trigger signal may also be generated by one or more keys on the keypad 46.

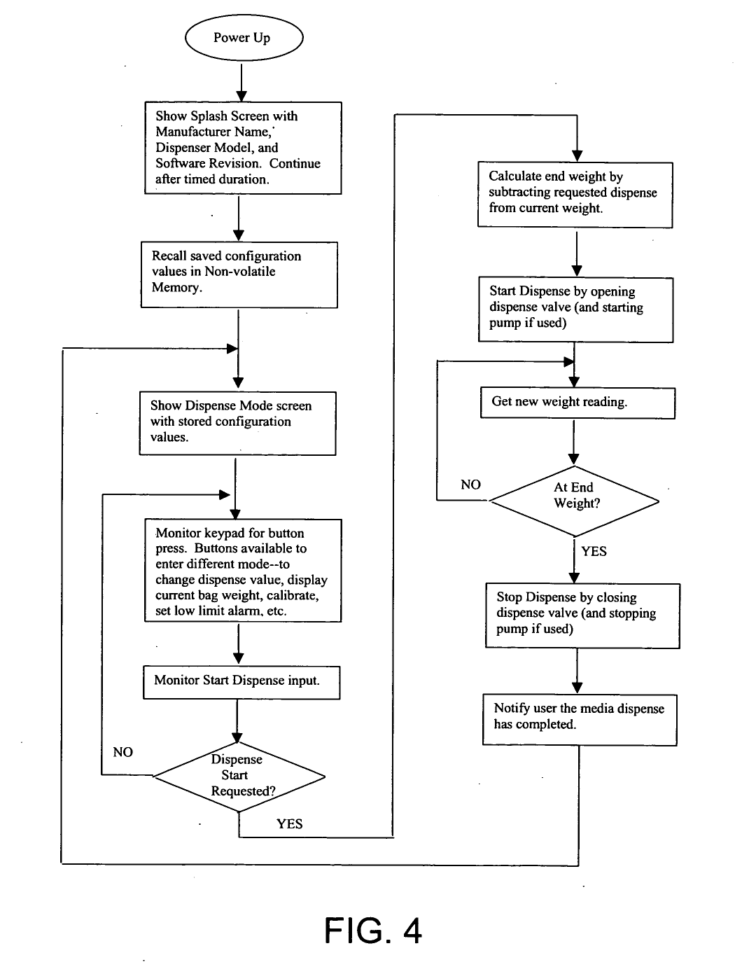

After receiving a trigger signal, and having been programmed with the volume of the aliquot, the controller 26 calculates the target weight (“target count”) of the bag 14 by subtracting the weight of the aliquot (“dispense count”) from the current weight of the bag 14. The controller 26 then opens the pinch valve 28, which allows media to flow through the dispense tube 20. While media is flowing, the controller 26 continuously monitors the decreasing weight of the bag 14 by continuously sampling the load cell output signal. When the target weight is reached, the controller 26 closes the pinch valve 28 and interrupts fluid dispense. A controller logic diagram is shown in FIG. 4.

Typical load cells of the size described above generate weak output signals. For example, the load cell described above develops an output signal of about 2 mV per volt of applied excitation over its rated load capacity of 2000 g. With an applied excitation of 10V, for example, the range of output signal is only about 0 to 20 mV. Since the value of the load cell output signal is low, the controller 26 must be designed to minimize electrical noise, which may sharply compromise the load cell output signal and compromise the accuracy of the weight-detecting function of the load cell 24. Further, because of the sensitivity of the load cell 24, the housing 12 should be securely mounted to a stationary object or located in a location remote from physical vibration or other disturbances, so that additional external forces do not act on the bag 14 and compromise the accuracy of the weight-detecting function of the load cell 24.

The weak output signal of the load cell 24 must be amplified because microprocessors typically require input signals significantly greater than the output signal typically generated by a load cell. For example, the microprocessor described above requires an input signal strength from 0 to about 4 V, whereas the load cell output signal described above is from 0 to 20 mV. Therefore, the controller 26 may include an amplifier, which, in this example, should amplify the output of the load cell 24 by a factor of about 200. However, to account for overload in the bag 14, i.e., more than 2000 g fluid in the bag, the gain of the amplifier may be limited to a lower factor of, for example, 187.5, which would result in a maximum voltage of 3.75V when the bag contains 2000 g fluid. In a preferred embodiment, the amplifier comprises a precision instrumentation amplifier integrated circuit (IC), which is integrated with the analog-to-digital converter, described below.

To convert the load cell signal (now amplified) from analog to digital, the controller includes an analog-to-digital converter (ADC) 25. In preferred embodiments, analog to digital conversion of the load cell output signal is performed by either a successive-approximation (SAR) ADC IC or a sigma-delta ADC IC. In the embodiment illustrated in FIGS. 1-4, the controller 24 includes a bridge transducer analog-to-digital converter (ADC) 25, model AD7730, manufactured by Analog Devices, Inc., which includes a 24 bit resolution sigma-delta ADC and an amplifier. In either case, an inherent compromise exists between the resolution of the ADC and its speed of conversion. For example, a high-performance SAR ADC having 16-bit resolution may be used. With 16-bit resolution, the ADC has a range of 65,536 counts (2¹⁶) with 61,440 counts (93.75%) being in the working range of the bag 14. In this embodiment the volume of liquid dispensed could be controlled to the 0.0325 g (2000 g/61,440). Accordingly, assuming the density of the cell culture media was equal to 1, a dispense count of 31 (0000000000011111) would approximately equate to 1 g or 1 ml, and a dispense count of 154 (0000000010011010) would approximately equate to 5 g or 5 ml.

As described above, noise may also affect the accuracy of the load cell output signal. Accordingly, rather then relying on a single value, the SAR ADC may calculate multiple values and average the values. The averaging of values acts as a filter as numbers that were affected by noise will have less impact. Increasing the number of values that are used improves filtering but adversely affects the time required to record a change in the weight of the container 14. For example, the SAR ADC may maintain the numbers in a circular queue that has a power of two queues (e.g., 8, 16, 32) and the average is the average of the numbers stored in the queues (rolling average).

By increasing the number of samples in the queue, the degree of filtering is improved, but at the expense of a reduction in the speed of response to changes in the measured weight of the container 14. Therefore, a relatively fast response is required in order to close the pinch valve 28 promptly and without over-dispensing. For example, if a fine tip dispenser 34 is used to dispense media at a rate of 1 ml/S, then a delay of 10 mS in recognizing the target weight or count would result in an over-dispense of 1%. Therefore, the filtered data should preferably be able to track the actual signal at better than 100 Hz. Variations of the moving-average scheme in which the older samples are weighted to reduce their contribution over time are also possible.

Alternatively, rather than using a rolling average, a burst average may be used. The burst average calculates an average for every certain number of calculations (e.g., 8, 16, 32) and loads these values into a circular queue that calculates the rolling average of the bursts of calculations.

According to another embodiment, a sigma-delta ADC may be used that has 24 bit resolution so that filtering is built in the conversion. A sigma-delta ADC offers much higher resolution (typically, 24-bits) than a SAR ADC but has a much lower conversion rate. The sigma-delta ADC performs the same function as the SAR ADC except that the software filtering is replaced largely by the hardware filtering inherent in the sigma-delta architecture.

The controller 26 may be programmed to assume that the density of the media is 1, and thus equivalence exists between weight and volume. Alternatively, the controller 26 may be programmed with the specific density of the fluid contained within the bag 14, which is then used to calculate the weight of the aliquot. Alternatively, the weight of the aliquot can be calibrated using a volumetric measuring cylinder or electronic scale. This calibration procedure may also be used to compensate for any response lag of the control valve.

In a preferred embodiment, the container 14 and dispense tube 20 are integrally formed. The bag 14 is filled and packaged in a sterile environment. The free end of the tube 20 has a seal, such as a disposable, manual pinch valve, which is removed after the dispense tube is connected to the pinch valve 28.

To reduce the cost of the bag 14 and dispense tube 20, the pinch valve 28 is preferably located inside the housing 12 to reduce the length of silicone or C-flex dispense tubing needed to pass through the pinch valve 28 and exit the housing 12. Thereafter, an extension of less expensive flexible tubing 32 may be connected to the dispense tube 20 by, for example, a luer coupling 30. The extension tubing 32 connects at the other end to the dispense tip 34. If the dispense tip 34 or extension tubing 32 becomes contaminated, either can be replaced without replacing or disturbing the bag 14.

In the embodiment shown in FIGS. 1-4, media is dispensed from the bag primarily by the force of gravity. Consequently, the housing 12 and bag 14 must be positioned at a location higher than the bench top 4 and cell culture containers 6. However, in this embodiment, the bag 14 or other container need not be flexible or compressible.

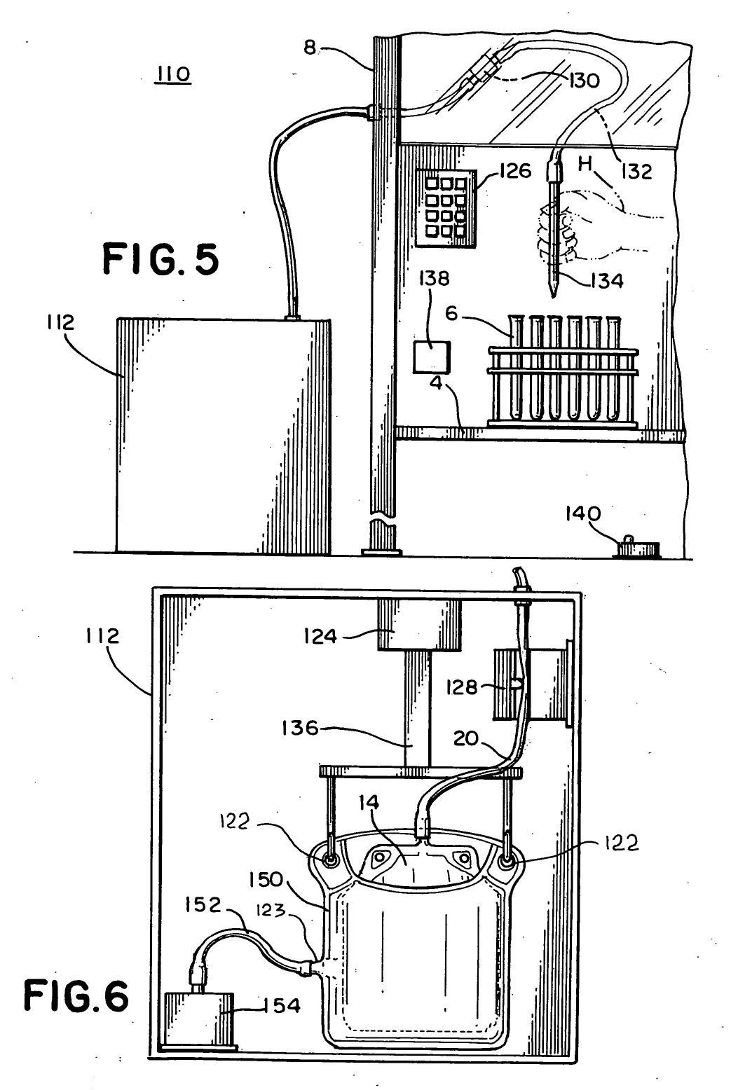

In another embodiment of the invention shown in FIGS. 5-6, the dispenser 110 can dispense fluid media to heights above the housing. The dispenser 110 has a construction similar to the dispenser 10 described above; however, the dispenser 110 includes a container pressurizer, which applies a compressive force on the outer surface of the bag 14 and increases fluid flow from bag 14. Therefore, the housing 112 and bag 14 need not be positioned at a location higher than the bench top 4 of the laminar flow hood 8 or cell culture containers 6.

In this embodiment, the container pressurizer comprises a double-walled compression sleeve 150 having a closed bottom and an open top. The sleeve 150 includes two opposed eyelets 122 formed in the upper perimeter, which engage hooks on the load cell support bracket 136 and which suspend the sleeve 150 from the load cell 124. The bag 14 is inserted in and supported by the compression sleeve 150.

A port 123 is formed in the sidewall of the compression sleeve 150. The port 123 connects to a tube 152, which provides fluid communication between a pump 154 and the interior of the sleeve 150. The pump 154 is functionally connected to a controller 126, which activates and deactivates the pump 154 as needed to expel fluid from the container 14. In the embodiment shown in FIGS. 5 and 6, the controller 126 is housed within a keypad 146. A pinch valve 128, luer coupling 130, disposable flexible tube 132, dispense tip 134, trigger switch 138 and foot switch 140 are similar to their respective components described above. Generally, with the exception of the compression sleeve 150 and air pump 154, the dispenser 110 has a similar construction and operates in a manner similar to the dispenser 10 described above and shown in FIGS. 1-4.

While a compression sleeve 150 is disclosed, it should be appreciated that other means may be employed to pressurize the contents of the bag 14. For example, the bag 14 itself may have sufficient compressive elasticity to compress and expel media therefrom if initially pressurized. Alternatively, an elastic sleeve with sufficient compressive elasticity to compress the bag and expel media therefrom, or as described below, a sealed, pressurized housing may be used.

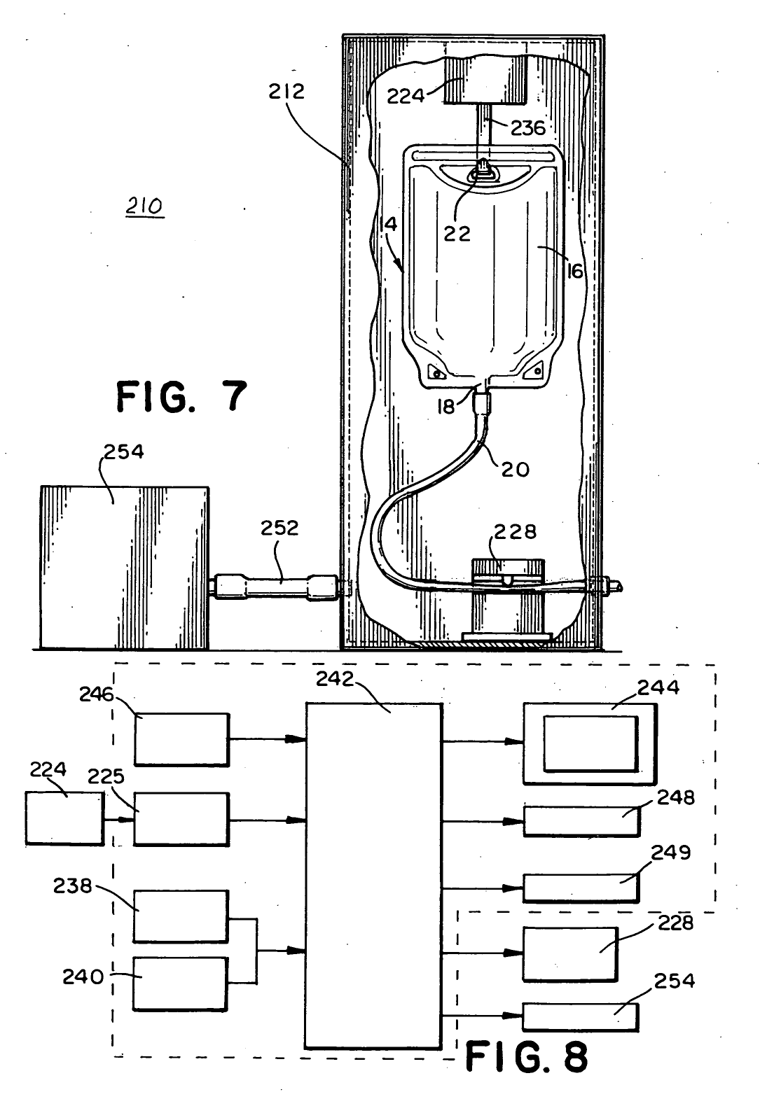

In another embodiment of the invention 210 shown in FIGS. 7 and 8, the dispenser 210 has a construction similar to the dispenser 10 described above; however, the dispenser 210 has a fully-enclosed housing 212 and an air pump 252 in fluid communication with the housing 212. In this embodiment, the housing 212 of the dispenser 210 can be either positively or negatively pressurized increase fluid flow from the bag 14 or enable media infusion into the bag 14.

Referring to FIGS. 7 and 8, the housing 212 is fully-enclosed and connected in fluid communication by a tube 252 to a pump 254, which can either admit or evacuate air from the housing 212. The bag 14 suspends from a hook 236 on the load cell 224. In this embodiment, elevated air pressure in the housing 212 applies a compressive force on the bag 14 in the manner and for the purposes described above with respect the embodiment shown in FIGS. 5-6. However, in this embodiment, a vacuum may also be created in the housing 212, which enables media to be drawn inwardly through the dispense tube 20 and infused into the container 14.

The pump 254 is functionally connected to the controller 226, which activates and deactivates the pump 254 as needed to expel fluid from or admit fluid to the bag 14. In the embodiment shown in FIGS. 7 and 8, the pinch valve 228, luer coupling 230, disposable flexible tube 232, dispense tip 234, trigger switch 238, foot switch 240, LCD display 244, keypad 246 are similar to their respective components described above. In this embodiment, the controller 226 also includes LED's 228 and a buzzer 248 to alert the user to predefined conditions. The dispenser 210 has a similar construction and operates in a manner similar to the dispensers 10 and 110 described above.

The invention also provides a method of feeding cell cultures in a laminar flow hood. The method of feeding reduces the risk of contaminating the cell culture media and reduces the amount of disposable media container waste.

Cell culture media is initially produced in a very large container such as, for example, a 20 liter bag. The media is then apportioned into the 2-liter disposable, flexible bags described above. A 2-liter bag is loaded into a dispenser as described above. The dispense tube is threaded through the pinch valve of the dispenser. The free end of the dispense tube is positioned within the hood. The disposable extension tube and dispense tip are then connected to the dispense tube. The volume of the aliquot is programmed into the controller. Aliquots of media are then dispensed to the cells by activating one of the trigger switches.

If the dispense tip becomes contaminated, the tip is discarded and replaced. Likewise, if the extension tube is contaminated, it is discarded and replaced. Cells are fed until the media contained in the bag is exhausted. By using the aforementioned apparatus and method, the risk of contamination of the cell culture media is reduced. Further, the amount of disposable media container waste is also significantly reduced.

While the principles of the invention have been described above in connection with specific embodiments, it is to be clearly understood that this description is made only by way of example and not as a limitation on the scope of the invention.

1. An apparatus for dispensing precise aliquots of cell culture media from a flexible container having a flexible dispense tube and a maximum volume V, comprising:
a) a housing;
b) a programmable controller;
c) a load cell fixed to said housing and functionally transmitting a load cell output signal to said controller;
d) a support which releasably suspends the container from the load cell;
e) a control valve functionally connected to said controller, said control valve regulating the flow of media through the dispense tube; and,
wherein said controller measures the volume of media dispensed from the container by continuously calculating the change in weight of the container using the load cell output signal.

2. The device recited in claim 1, wherein the control valve engages only the outer surface of the flexible dispense tube and does not contact the cell culture media.

3. The device recited in claim 1, wherein said control valve comprises a solenoid-actuated pinch valve.

4. The apparatus recited in claim 1, wherein media is dispensed from the container opening the control valve and allowing gravity to force media from the container.

5. The apparatus recited in claim 1, including a container pressurizer for increasing media flow from the container.

6. The apparatus recited in claim 5, wherein said container pressurizer comprises a pump and pneumatic compression sleeve surrounding and compressing the container.

7. The apparatus recited in claim 6, wherein the pump is functionally connected to said controller.

8. The apparatus recited in claim 6, wherein said compression sleeve is connected to said load cell and suspends the container from said load cell.

9. The apparatus recited in claim 5, wherein said load cell and container are contained in a fully-enclosed housing, and said housing is pressurized by a pump, which is functionally connected to said controller.

10. The apparatus recited in claim 9, wherein said pump can create either positive or negative pressure in said housing for either increasing media media flow from the container or infusing media into the container.

11. The apparatus recited in claim 1, wherein said controller can be programmed to dispense aliquots of any volume less than or equal to V.

12. The apparatus recited in claim 1, wherein said controller can be calibrated with the specific density of the media contained within the container.

13. The apparatus recited in claim 1, wherein said controller includes a digital microprocessor, an amplifier, an analog-to-digital converter (ADC), and a dispense trigger.

14. The apparatus recited in claim 13, wherein the ADC comprises a successive-approximation (SAR) ADC amplifier integrated circuit.

15. The apparatus recited in claim 13, wherein the ADC comprises a sigma-delta ADC amplifier integrated circuit.

16. The apparatus recited in claim 1, including means for preventing contamination of the media in the container during dispensing.

17. The apparatus recited in claim 16, including a flexible extension tube releasably fixed to the free end of the dispense tube of the container, and a disposable pipet releasably fixed to the end of said extension tube.

18. A method of dispensing precise aliquots of cell culture media from a sterile media container having a flexible dispense tube at the bottom end, comprising:
a) suspending the container from a load cell;
b) weighing the container;
c) calculating the target weight of the container after the aliquot is dispensed;
d) initiating media dispense from the container;
e) continuously monitoring the current weight of the container;
f) terminating media dispense from the container once the current container weight equals the target weight.

19. The method recited in claim 18, wherein media dispense is initiated and terminated by intermittently restricting flow through the flexible dispense tube.

20. The method recited in claim 19, including pressuring the container to increase fluid flow from or into the container.

21. A method of feeding cell cultures in a laminar flow hood, comprising the steps of:
a) providing a flexible bag of cell culture media, said bag having a storage reservoir, an outlet at the bottom end, and a flexible dispense tube connected to the outlet;
b) providing a cell culture media dispenser having:
i) a housing;
ii) a programmable controller;
iii) a load cell fixed to said housing and functionally transmitting a load cell output signal to said controller;
iv) a support which releasably suspends the container from the load cell; and,
v) control valve functionally connected to said controller, said control valve regulating the flow of media through the dispense tube;
wherein said controller measures the volume of media dispensed from the container by continuously calculating the change in weight of the container using the load cell output signal;
c) loading the bag of cell culture media on the load cell;
d) connecting the dispense tube of the bag to the control valve;
e) connecting the free end of the dispense tube to a disposable pipet;
f) programming the controller with the volume of the aliquot; and,
g) activating the controller to dispense precise aliquots of cell culture media.
